(19) United States
(12) Patent Application Publication
(10) Pub. No.: US 2003/0196281 A1
(43) Pub. Date: Oct. 23, 2003

(54) 1-benzopyrane-derivatives and colouring agents containing the salts thereof

(76) Inventors: Guido Sauter, Thoerishaus (CH); Hans-Juergen Braun, Uebersdorf (CH); Raymond Brouillard, Strausborg (FR); Andre Fougerousse, Birkenwald (FR); Christine Roehi-Stoeckel, Strasbourg (DE)

Correspondence Address: Striker Striker & Stenby, 103 East Neck Road, Huntington, NY, 11743, US

(21) Appl. No.: 10/380,896

(22) PCT Filed: Feb. 6, 2002
(86) PCT No.: PCT/EP02/01194

(30) Foreign Application Priority Data
Jun. 22, 2001 (DE) 101 30 144.8

(51) Int. Cl. A61K 7/13

(52) U.S. Cl. 8/405; 8/406

(57) ABSTRACT

The present invention concerns an agent for coloring fibers that is obtained by mixing two components—if necessary with the addition of an alkalinizing agent or an acid—said agent being characterized in that one component (component A2) contains at least one electrophilic compound and the other component (component A1) contains at least one 1-benzopyran of formula (Ia)/(Ib) or a 1-benzopyrylium derivative of formula (I),

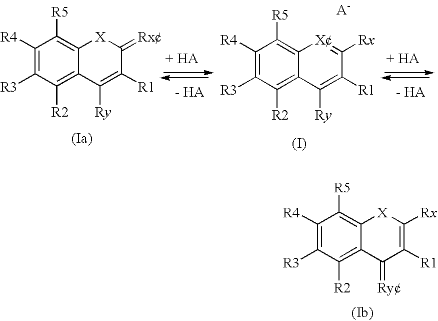

and a method for temporarily coloring keratin fibers whereby the hair is first colored with the aforesaid coloring agent and then, at any future time, is again decolorized with a reducing agent.

The present invention relates to colorants, particularly for keratin fibers, for example human hair, which contain a 1-benzopyran derivative and/or a 1-benzopyrylium derivative with acidic CH groups in the 2- and/or 4-positions.

In general, fibers, for example hair, wool or furs, are dyed either with direct dyes or with oxidation dyes in which case the coloration is obtained by reaction of certain developers with certain couplers in the presence of an appropriate oxidant. Although the oxidation dyes give intense colorations with good fastness properties, the development of the color is the result of the action of oxidants which in some cases can cause damage to the fibers. Moreover, some oxidation dye precursors can, in exceptional cases, cause allergies in predisposed persons. Direct dyes are applied under more gentle conditions, but their drawback is that the colorations often do not have adequate fastness properties.

European Patent EP 0 847 749 discloses the use of a combination of diiminoisoindoline derivatives or 3-aminoisoindolone derivatives with compounds having primary or secondary amino groups for dyeing keratin fibers without the addition of oxidants. Moreover, it is known from German Unexamined Patent Application DE 43 35 623 to use for dyeing keratin fibers a combination of indolinone derivatives and compounds with primary or secondary amino groups, heterocyclic compounds or aromatic hydroxy compounds. German Unexamined Patent Application 44 09 143 describes the use of satin derivatives for dyeing keratin fibers. Unexamined German Patent Application 197 45 292 discloses the use of a combination of malonaldehyde derivatives, for example malonaldehyde bisdialkylacetals, and amines or active CH compounds for dyeing hair without the addition of oxidants. Furthermore, it is known from Unexamined German Patent Application 197 17 280 to use a combination of certain heterocyclic aldehydes and amines or active CH compounds to dye hair without the addition of oxidants.

However, the agents and methods for dyeing fibers, known from the prior art, are not satisfactory in every respect. Hence, a great demand continued to exist for colorants which under mild conditions give both intense and gentle colorations in a wide range of color shades and—if desired—can readily be decolorized at a later date.

The object of the present invention therefore is to provide a coloring system which without the addition of an oxidant (for example hydrogen peroxide) gives rise to a gentle and at the same time intense fiber coloration in the yellow, brown, green and violet range with good fastness properties (light fastness, wash fastness, rubbing fastness) and which at any desired point in time can be removed in simple and gentle manner.

Surprisingly, we have now found that the use of a combination of (a) certain 1-benzopyran derivatives and/or 1-benzopyrylium derivatives of formula (I)/(Ia)/(Ib) which in the 2- and/or 4-position are substituted with an acidic CH group and (b) at least one electrophilic compound affords in gentle manner colorations with outstanding brilliance and color depth and which later, at any desired time, can be readily and completely removed.

The object of the present invention therefore is an agent for coloring fibers, for example cotton, jute, sisal, linen or modified natural fibers, for example regenerated cellulose, nitrocellulose, alkylcellulose, hydroxyalkylcellulose or acetylcellulose, or synthetic fibers, for example polyamide fibers, polyacrylonitrile fibers, polyurethane fibers, polyester fibers, and especially keratin fibers, for example wool, silk or hair—particularly human hair—which is obtained by mixing two components—if necessary with addition of an alkalinizing agent or an acid—and which is characterized in that one component (component A2) contains at least one electrophilic compound and the other component (component A1) contains at least one 1-benzopyran of formula (Ia) or formula (Ib) or a 1-benzopyrylium derivative of formula (I),

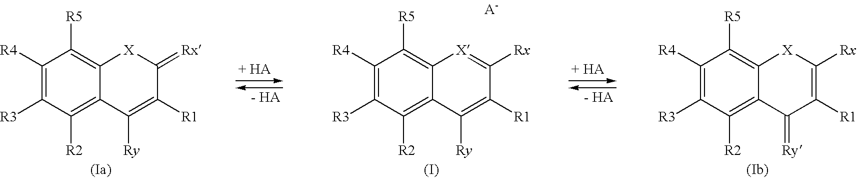

wherein:

X denotes an oxygen atom, sulfur atom or selenium atom;

Rx and Ry independently of each other denote a hydrogen atom, an aryl group, a C₁-C₈-alkyl group, a C₁-C₈-monohydroxyalkyl group, a C₁-C₈-polyhydroxyalkyl group, a C₁-C₈-alkoxy-(C₁-C₈)-alkyl group, a halogen atom (F, Cl, Br, I), an —O—COR^{a} group, an —S—COR^{a} group, an SR^{a} group, a heteroaryl group, a phenyl group, a benzyl group, an —OCH₂-aryl group or a —CH₂-Rz group;

Rx′ and Ry′ independently of each other denote a —CH-Rz group;

Rz denotes a hydrogen atom, an optionally substituted phenyl group, an optionally substituted naphthyl group, an optionally substituted aromatic carbon ring or heterocyclic ring, a C₁-C₈-alkyl group, a C₁-C₈-monohydroxyalkyl group, a C₁-C₈-polyhydroxyalkyl group, a C₁-C₈-alkoxy-(C₁-C₈)-alkyl group, a halogen atom (F, Cl, Br, I), an —OCOR^{a} group, a nitro group, a cyano group, a —CO-R^{a} group, a —CO-OR^{a} group, a —CO—OCF₃ group, a —CONHR^{a} group, a —CO—N(R^{a})₂ group, an —SO₂—NH₂ group, an —SO₂—NHR^{a} group, an —SO₂—N(R^{a})₂ group or an —SO₂—OR^{a} group, wherein R^{a} stands for a hydrogen atom, an optionally substituted aromatic carbon ring or heterocyclic ring or a (C₁-C₆)-alkyl group; provided that the tautomeric formula (Ia) is valid only when Rx is a —CH₂-Rz group, and the tautomeric formula (Ib) is valid only when Ry is a —CH₂-Rz group, and Rz in Rx and Rx′ or in Ry and Ry′ always has the same meaning; R1, R2, R3, R4 and R5 independently of each other denote a hydrogen atom, a straight-chain or branched (C₁-C₄)-alkyl group, a (C₁-C₄)-hydroxyalkyl group, a hydroxyl group, a methoxy group, a benzyl group, a halogen atom (F, Cl, Br, I), a nitro group, a nitroso group, a cyano group, a trifluoromethyl group, a —CHO group, a —COR^{b} group, a —COOH group, a —CO₂R^{b} group, an —OCOR^{b} group, an —OCH₂-aryl group, an —SO₂NH₂ group, an —SO₂CHF₂ group, an —SO₂CF₃ group, an —SO₂NH₂ group, an —SO₂NHR^{b} group, an —SO₂N(R^{b})₂ group, an —SO₂R^{b} group, an —NH₂ group, an —(NH₃)⁺ group, an —NHR^{b} group, an —(NH₂R^{b})⁺ group, an —N(R^{b})₂ group, an —[N(R^{b})₃]⁺ group, an —NHCOR^{b} group, an —NHCOOR^{b} group, a CH₂NH₂ group, a —CH₂NHR^{b} group, a —CH₂N(R^{b})₂ group, a —CO₂CF₃ group or a —PO(OR^{b})₂ group, wherein R^{b} stands for a hydrogen atom, an optionally substituted aromatic carbon ring or heterocyclic ring or a (C₁-C₆)-alkyl group); and

A⁻ denotes an anion of an organic or inorganic acid, preferably a chloride, bromide, iodide, hydrogen sulfate, sulfate, toluenesulfonate, benzenesulfonate, mono-methylsulfate, hexafluorophosphate, hexafluoroantimonate, tetrafluoroborate, tetraphenylborate, formate, acetate or propionate ion, with the chloride ion, tetrafluoroborate ion, acetate ion and hydrogen sulfate ion being particularly preferred. The compounds of formula (I) wherein Rx and/or Ry denote —CH₂-Rz, with Rz denoting hydrogen, are preferred.

Suitable compounds of formulas (I), (Ia) and (Ib) are, in particular 7-hydroxy-4-methylene-2-phenyl-4H-1-benzopyran; 7-methoxy-4-methylene-2-(3,4,5-trimethoxyphenyl)-4H-1-benzopyran; 5,7-dihydroxy-4-methylene-2-phenyl-4H-1-benzopyran; 5,7-dimethoxy-4-methylene-2-phenyl-4H-1-benzopyran; 4-methylene-4H-1-benzopyran; 4-methylene-2-phenyl-4H-1-benzopyran; 7-amino-4-methylene-2-phenyl-4H-1-benzopyran; 7-dimethylamino-4-methylene-2-phenyl-4H-1-benzopyran; 4-methylene-7-nitro-2-phenyl-4H-1-benzopyran; 2-methylene-2H-1-benzopyran, 2-methylene-4-phenyl-2H-1-benzopyran; 2-methylene-4-methyl-2H-1-benzopyran and 4-methylene-2-methyl-4H-1-benzopyran and the salts thereof, particularly preferred being 7-hydroxy-4-methyl-2-phenyl-1-benzopyrylium chloride, 7-methoxy-4-methyl-2-(3,4,5-trimethoxyphenyl)-1-benzopyrylium chloride, 7-methoxy-4-methyl-2-(3,4,5-trimethoxyphenyl)-1-benzopyrylium chloride, 5,7-dihydroxy-4-methyl-2-phenyl-1-benzopyrylium chloride, 5,7-dimethoxy-4-methyl-2-phenyl-1-benzopyrylium chloride, 4-methyl-1-benzopyrylium chloride, 4-methyl-2-phenyl-1-benzopyrylium chloride, 7-amino-4-methyl-2-phenyl-1-benzopyrylium chloride, 7-dimethylamino-4-methyl-2-phenyl-1-benzopyrylium chloride, 4-methyl-7-nitro-2-phenyl-1-benzopyrylium chloride, 2-methyl-1-benzopyrylium chloride, 2-methyl-4-phenyl-1-benzopyrylium chloride, 7-hydroxy-2,4-dimethyl-1-benzopyrylium chloride, 5,7-dihydroxy-2,4-dimethyl-1-benzopyrylium chloride, 7-dimethylamino-2,4-dimethyl-1-benzopyrylium chloride and 2,4-dimethyl-1-benzopyrylium chloride.

According to the present invention, by “electrophilic compounds” are meant compounds which by reaction on the nucleophilic carbon of the —CH-Rz group in Rx′ or Ry′ in formula (Ia) or formula (Ib) can form unsaturated carbon-carbon bonds. Suitable electrophilic compounds therefore contain an electrophilic reaction center, for example a carbonyl carbon, an imine carbon or an sp²-hybridized carbon atom activated by a heteroatom. Examples of suitable electrophilic compounds are, in particular, carbonyl compounds, imino compounds and substituted 1-alkylquinolinium derivatives of formulas (IIa) and (IIb)

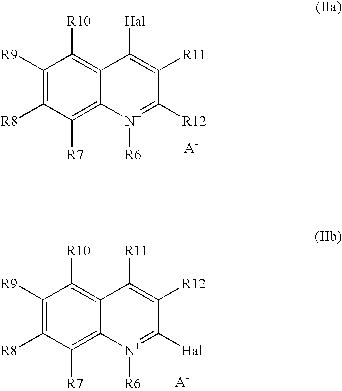

wherein the R6 group denotes a straight-chain or branched C₁-C₈-alkyl group, a C₁-C₈-monohydroxyalkyl group, a C₂-C₈-polyhydroxyalkyl group, a C₁-C₈-alkoxy-(C₁-C₈)-alkyl group of a thio-(C₁-C₈)-alkyl group;

the R7, R8, R9, R10, R11 and R12 groups independently of each other denote a hydrogen atom, a straight-chain or branched C₁-C₄-alkyl group, a straight-chain or branched C₁-C₄ hydroxyalkyl group, a hydroxyl group, a methoxy group, an ethoxy group, a benzyl group, a halogen atom (F, Cl, Br, I), a nitro group, a nitroso group, a cyano group, a trifluoromethyl group, a —CHO group, a —COR^{c} group, a —COOH group, a —CO₂R^{c} group, an —OCOR^{c} group, an —O—SO₂CF₃ group, an —OCH₂-aryl group, an —SO₂NH₂ group, an —SO₂CHF₂ group, an —SO₂CF₃ group, an —SO₂NH₂ group, an —SO₂NHR^{c} group, an —SO₂N(R^{c})₂ group, an —SO₂R^{c} group, an —NH₂ group, an —(NH₃)⁺ group, an —NHRC group, an —(NH₂R^{c})⁺ group, an —N(R^{c})₂ group, an —[N(R^{c})₃]⁺ group, an —NHCOR^{c} group, an —NHCOOR^{c} group, a —CH₂NH₂ group, a —CH₂NHR^{c} group, a CH₂N(R^{c})₂ group, a —CO₂CF₃ group or a —PO(OR^{c})₂ group, wherein R^{c} stands for a hydrogen atom, an optionally substituted aromatic carbon ring or heterocyclic ring or a (C₁-C₆)-alkyl group);

Hal denotes an iodine atom, bromine atom, chlorine atom or a methoxy group, ethoxy group, CF₃—SO₂—O— group, aryl-SO₂—O— group or (SO₃)— group, with a chlorine atom and ethoxy group being preferred, and A⁻ having the afore-indicated meaning.

The compounds of formulas (IIa) and (IIb) are known from the literature and can be prepared, for example, by standard methods of synthesis, for example as described in U.S. Pat. No. 3,149,105 and British Patent 1 102 891 or by T. G. Deligeorgiev et al. in Dyes and Pigments 1999, pp. 49-54 and by G. B. Barlin et al. in J. Chem. Soc. Perkin Trans. 2, 1975, pp. 298-302.

Suitable carbonyl compounds and imino compounds are, in particular, 4-hydroxy-3-methoxybenzaldehyde (vanillin), 3-hydroxy-4-methoxybenzaldehyde (isovanillin), 3,4-dihydroxybenzaldehyde,4-hydroxybenzaldehyde,3,5-dimethoxy-4-hydroxybenzaldehyde, 4-dimethylaminobenzaldehyde, 4-methyl-5-imidazolcarboxaldehyde, 4-dimethylaminocinnamaldehyde, 4-hydroxy-2-methoxybenzaldehyde, 3,5-dimethyl-4-hydroxybenzaldehyde, 4-dimethylamino-2-methoxybenzaldehyde, 2-hydroxybenzaldehyde, 4-hydroxy-1-naphthaldehyde, 4-methoxy-1-naphthaldehyde, 4-dimethylamino-1-naphthaldehyde, 4′-hydroxybiphenyl-1-carbaldehyde, 2-hydroxy-3-methoxybenzaldehyde, 2,4-dihydroxybenzaldehyde, 3,4-dihydroxybenzaldehyde, 2,5-dihydroxybenzaldehyde, 2,3,4-trihydroxybenzaldehyde, 3,4,5-trihydroxybenzaldehyde, 2,4,6-trihydroxybenzaldehyde, 2,4-dimethoxybenzaldehyde, 2,3-dimethoxybenzaldehyde, 2,5-dimethoxybenzaldehyde, 3,5-dimethoxybenzaldehyde, 3,4-dimethoxybenzaldehyde, indole-3-carbaldehyde, benzene-1,4-dicarbaldehyde,-4-ethoxybenzaldehyde, 2-methyl-1,4-naphthoquinone, 4-carboxybenzaldehyde, 4-hydroxy-3-methoxycinnamaldehyde, 3,5-dimethoxy-4-hydroxycinnamaldehyde, 3-methoxy-4-(1-pyrrolidinyl)-benzaldehyde, 4-diethylamino-3-methoxybenzaldehyde, 1,2-phthalic dialdehyde, pyrrole-2-aldehyde, thiophene-2-aldehyde, thiophene-3-aldehyde, chromone-3-carboxaldehyde, 6-methyl-4-keto-1 (4H)benzopyran-3-carbaldehyde, N-methylpyrrole-2-aldehyde, 5-methylfurfural, 6-hydroxychromene-3-carboxaldehyde, 6-methylindole-3-carboxaldehyde, 4-dibutylaminobenzaldehyde, N-ethylcarbazole-3-aldehyde, 4-diethylamino-2-hydroxybenzaldehyde, 3,4-dimethoxy-5-hydroxybenzaldehyde, 5-[4-(diethylamino)phenyl]-2,4-pentadienal, 2,3-thiophenedicarboxaldehyde, 2,5-thio-phenedicarboxaldehyde, 2-methoxy-1-naphthaldehyde, 3-ethoxy-4-hydroxybenzaldehyde, 2-nitrobenzaldehyde, 3-nitrobenzaldehyde and 4-nitrobenzaldehyde, 4-{[(2-hydroxyethyl)imino]methyl}-2-methoxyphenol, 5-{[(2-hydroxyethyl)imino]methyl}-2-methoxyphenol, 2,6-dimethoxy-4-{[(2-hydroxyethyl)imino]methyl}phenol, 4-{[(2-hydroxyethyl)imino]methyl}phenol, 1, 2-dihydroxy-4-{[(2-hydroxyethyl) imino]methyl}benzene, N,N-dimethyl-4-{[(2-hydroxyethyl)imino]methyl}aniline, 1,2-dihydroxy-3-{[(2-hydroxyethyl)imino]methyl}benzene, 4-{[(3-hydroxypropyl)imino]methyl}phenol, 2,6-dimethoxy-4-{[(3-hydroxypropyl)imino]methyl}phenol, 4-{[(2,3-dihydroxypropyl)imino]methyl}phenol, 2,6-dimethoxy-4-{[(2,3-dihydroxypropyl)imino]methyl}phenol, 2-[(4-hydroxybenzylidene)amino]propane-1,3-diol, 2-[(4-hydroxy-3,5-dimethoxybenzylidene)amino]propane-1,3-diol, 4-{[(2-hydroxy-2-phenylethyl)imino]-methyl}phenol, 2,6-dimethoxy-4-{[(1-phenyl-2-hydroxyethyl)imino]methyl}phenol, 4-{[(2-hydroxyphenyl)imino]methyl}phenol, 2,6-dimethoxy-4-{[(2-hydroxyphenyl)-imino]methyl}phenol, 5-guanidino-2-[(4-hydroxybenzylidene)amino]pentanoic acid, 2-[(4-dimethylaminonaphthalen-1-yl-methylene)aminolethanol, 5-guanidino-2-[(4-hydroxy-3,5-dimethoxybenzylidene)amino]pentanoic acid, 2-[(4-hydroxy-3,5-dimethoxybenzylidene)amino]-3-(3H-imidazol-4-yl)propanoic acid, 2-[(4-hydroxybenzylidene)amino]-3-(3H-imidazol-4-yl)-propanoic acid, 2-[(4-hydroxy-3,5-dimethoxybenzylidene)amino]-3-(1H-indol-3-yl)propanoic acid, 2-[(4-hydroxybenzylidene)amino]-3-(1H-indol-3-yl)propanoic acid, 2-{[(2-hydroxyethyl)imino]methyl}phenol, 1,2-dihydroxy-3-{[(2-hydroxyethyl)imino]methyl}benzene, 1, 2,3-trihydroxy-4-{[(2-hydroxyethyl)imino]methyl}benzene, 1,2,3,4-tetrahydroxy-5-{[(2-hydroxyethyl)imino]methyl}benzene and 1,2,4-trihydroxy-4-{[(2-hydroxyethyl)imino]methyl}benzene.

The carbonyl compounds can also be prepared in situ by enzymatic oxidation of the corresponding aryl alcohols and benzyl alcohols with the aid of one or more suitable oxidation enzymes.

Suitable aryl alcohols and benzyl alcohols are, for example, the following compounds: benzyl alcohol, 4-hydroxybenzyl alcohol, 4-hydroxy-3-methoxybenzyl alcohol (vanillyl alcohol), 3-hydroxy-4-methoxybenzyl alcohol (isovanillyl alcohol), 3,5-dimethoxy-4-hydroxybenzyl alcohol, 3,4-dihydroxybenzyl alcohol, 2-hydroxy-3-methoxybenzyl alcohol, 4-ethoxybenzyl alcohol, 4-carboxybenzyl alcohol, 2,5-dihydroxybenzyl alcohol, 2,4-dihydroxybenzyl alcohol, 2-hydroxybenzyl alcohol, 3,5-dimethoxy-4-hydroxybenzyl alcohol, 4-hydroxy-2-methoxybenzyl alcohol, 2,4-dimethoxybenzyl alcohol, 2,3-dimethoxybenzyl alcohol, 2,5-dimethoxybenzyl alcohol, 3,5-di-methoxybenzyl alcohol, 3,4-methylenedioxybenzyl alcohol, 3,4-dimethoxybenzyl alcohol, 3-ethoxy-4-hydroxybenzyl alcohol, 3,5-dimethyl-4-hydroxybenzyl alcohol, 3,4-dimethoxy-5-hydroxybenzyl alcohol, 3,4,5-trimethoxybenzyl alcohol, 2,4,6-trihydroxybenzyl alcohol, 3,4,5-trihydroxybenzyl alcohol, 2,3,4-trihydroxybenzyl alcohol, 3,5-ditert.butyl-4-hydroxybenzyl alcohol, 2-nitrobenzyl alcohol, 3-nitrobenzyl alcohol, 4-nitrobenzyl alcohol, 2-aminobenzyl alcohol, 3-aminobenzyl alcohol, 3-amino-4-methylbenzyl alcohol, 3,5-diaminobenzyl alcohol, 4-aminobenzyl alcohol, 4-dimethylaminobenzyl alcohol, 4-diethylamino-2-hydroxybenzyl alcohol, 4-diethylamino-3-methoxybenzyl alcohol, 4-dimethylamino-2-methoxybenzyl alcohol, 4-dibutylaminobenzyl alcohol, 3-methoxy-4-(1-pyrrolidinyl)benzyl alcohol, (4-methoxy-naphthalen-1-yl)methanol, (4-dimethylaminonaphthalen-1-yl)methanol, 2-(hydroxymethyl)-1-naphthol, 1-naphthalenemethanol, 2-naphthalenemethanol, (2-methoxy-naphthalen-1-yl)methanol, 4-hydroxymethylnaphthalen-1-ol, 4′-hydroxymethylbiphenyl-4-ol, (4-hydroxymethylphenyl)methanol, 4-(3-hydroxypropenyl)-2-methoxyphenol, 4-(3-hydroxypropenyl)-2,6-dimethoxyphenol, 3-(4-dimethylaminophenyl)-2-propen-1-ol, 5-4-(diethylaminophenyl)-2,4-pentadien-1-ol, thiophen-2-ylmethanol, (5-hydroxymethylthiophen-2-yl)methanol, thiophen-3-ylmethanol, (1H-pyrrol-2-yl)methanol, (1-methyl-1H-pyrrol-2-yl)methanol, (5-methylfuran-2-yl)methanol, (1H-indol-3-yl)methanol and (6-methyl-1H-indol-3-yl)methanol.

In principle, a suitable “oxidation enzyme” is any enzyme capable of catalyzing the oxidation of the alcohol to an aldehyde or ketone. Nonlimiting examples of such enzymes are the following: alcohol dehydrogenases (E.C. Classification 1.1.1.-), alcohol oxidases (E.C. Classification 1.1.2.- and 1.1.3.- and 1.1.99.-), flavinoxidases (E.C. Classification 1.2.—), laccases (E.C. Classification 1.4.—-), peroxidases (E.C. Classification 1.1 1.1.-), hydroxylases and monooxygenases (E.C. Classification 1.13.12.- and 1.13.99.-), with enzymes optimized (for example genetically modified) for the oxidation of the alcohols used being particularly preferred.

The enzyme is preferably used in an amount of 5-100 units per millimole of substrate (alcohol), one unit of enzyme activity being the amount of enzyme required to catalyze the oxidation of 1 micromole of alcohol per minute.

Suitable substituted 1-alkylquinolinium compounds of formulas (IIa) and (IIb) are, in particular, the following:

4-chloro-1-ethylquinolinium salts, 4,7-dichloro-1-ethylquinolinium salts, 4-chloro-1-ethyl-7-trifluoromethylquinolinium salts, 4-chloro-1-ethyl-6-nitroquinolinium salts, 4-chloro-1-methylquinolinium salts, 4-methoxy-1-methylquinolinium salts, 4-ethoxy-1-methylquinolinium salts, 4-ethoxy-1-ethylquinolinium salts, 4-iodo-1-methylquinolinium salts, 4-chloro-1-methyl-2-phenylquinolinium salts, 4-chloro-1-methyl-3-[(methylphenylamino)sulfonyl]quinolinium salts, 4-chloro-2-{{[4-(dimethylamino)phenyl]imino}methyl}-6-methoxy-1-methylquinolinium salts, 4-chloro-1-ethyl-3-[(phenylamino)-sulfonyl]quinolinium salts, 4-chloro-6-dimethylcarbamoyl-1-methylquinolinium salts, 4-chloro-1-ethyl-6-sulfamoylquinolinium salts, 4-chloro-1-ethyl-6-formylquinolinium salts, 4-chloro-1-ethyl-7-nitroquinolinium salts, 4-chloro-1-ethyl-7-methoxyquinolinium salts, 2-chloro-1-methylquinolinium salts, 2,6-dichloro-1-methylquinolinium salts and 2-chloro-1-methyl-4-trifluoromethanesulfonyloxyquinolinium salts, particularly preferred among these being 4-chloro-1-ethylquinolinium tetrafluoroborate, 4,7-dichloro-1-ethylquinolinium tetrafluoroborate, 4-chloro-1-ethyl-7-trifluoromethylquinolinium [sic—Translator], 4-chloro-1-ethyl-6-nitroquinolinium tetrafluoroborate, 4-chloro-1-methylquinolinium chloride, 4-methoxy-1-methylquinolinium tetrafluoroborate, 4-iodo-1-methylquinolinium iodide, 4-chloro-1-methyl-2-phenylquinolinium tetrafluoroborate, 4-chloro-1-methyl-3-[(methylphenylamino)sulfonyl]quinolinium methylsulfate, 4-chloro-2-{{14-(dimethylamino)phenyl]imino}methyl}-6-methoxy-1-methylquinolinium chloride, 4-chloro-1-ethyl-3-[(phenylamino)sulfonyl]quinoliniumtetrafluoroborate, 4-chloro-6-dimethylcarbamoyl-1-methylquinolinium tetrafluoroborate, 4-chloro-1-ethyl-6-sulfamoylquinolinium tetrafluoroborate, 4-chloro-1-ethyl-6-formylquinolinium tetrafluoroborate, 4-chloro-1-ethyl-7-nitroquinolinium tetrafluoroborate, 4-chloro-1-ethyl-7-methoxyquinolinium tetrafluoroborate, 2-chloro-1-methylquinolinium tetrafluoroborate, 2,6-dichloro-1-methylquinolinium tetrafluoroborate and 2-chloro-1-methyl-4-trifluoromethylsulfonyloxyquinolinium tetrafluoroborate.

The compounds of formulas (I)/(Ia)/(Ib) are kept separated from the electrophilic compound until shortly before use. As a rule therefore the colorant of the invention consists of two components, namely a dye carrier composition (A1) containing the compounds of formulas (I)/(Ia)/(Ib) and optionally direct dyes, and a second dye carrier composition (A2) containing the electrophilic compounds and optionally direct dyes. These two components are mixed just before use to give a ready-to-use colorant which is then applied to the fibers to be dyed. It is, of course, also possible for one or both components to consist of several individual components which are mixed with each other before use. Particularly preferred, however, is a 2-component kit consisting of an agent of component (A1) and an agent of component (A2).

Although to be more gentle to the fibers the aforesaid colorant is preferably used without the addition of an oxidant, the aforesaid colorant can readily be used in conjunction with an oxidant, for example when simultaneous bleaching of the fibers is desired or when common oxidation dye precursors are to be added to the colorant. The compounds of formulas (I)/(Ia)/(Ib) and the electrophilic compound are contained in the dye carrier compositions [component (A1) or component (A2)] in a total amount of about 0.02 to 20 wt. %, preferably 0.2 to 10 wt. %, so that the ready-to-use colorant obtained by mixing components (A1) and (A2) contains the compounds of formulas (I)/(Ia)/(Ib) and the electrophilic compound in a total amount of about 0.01 to 10 wt. % and preferably 0.1 to 5 wt. %.

Moreover, the colorant of the invention can also contain common, physiologically harmless, direct dyes from the group of acidic or basic dyes, nitro dyes, azo dyes, quinone dyes and triphenylmethane dyes.

The direct dyes can be contained in component (A1) and/or component (A2) in a total amount of about 0.02 to 20 wt. %, preferably 0.2 to 10 wt. %, so that the total amount of direct dyes in the ready-to-use colorant obtained by mixing components (A1) and (A2) is about 0.01 to 10 wt. % and preferably 0.1 to 5 wt. %.

The ready-to-use colorant and components (A1) and (A2) can be formulated to be in the form of, for example, a solution, particularly an aqueous or aqueous-alcoholic solution. Another suitable form is a cream, gel, foam or emulsion. Such forms consist of a mixture of compounds of formulas (I)/(Ia)/(Ib) and/or the electrophilic compounds with additives commonly used for such preparations.

Common additives to solutions, creams, emulsions, gels or foams are, for example, solvents such as water, lower aliphatic alcohols, for example ethanol, n-propanol and isopropanol, or glycols such as glycerol and 1,2-propanediol; moreover wetting agents or emulsifiers from the classes of anionic, cationic, amphoteric or nonionic surfaceactive substances such as the fatty alcohol sulfates, ethoxylated fatty alcohol sulfates, alkyl sulfonates, alkylbenzenesulfonates, alkyltrimethylammonium salts, alkylbetaines, ethoxylated fatty alcohols, ethoxylated nonylphenols, fatty alkanolamides, ethoxylated fatty alcohols, ethoxylated nonylphenols, fatty alkanolamides [sic—Translator], ethoxylated fatty esters, furthermore thickeners such as the higher fatty alcohols, starch or cellulose derivatives, perfumes, hair pretreatment agents, conditioners, hair-swelling agents, preservatives, furthermore vaseline, paraffin oil and fatty acids as well as hair-care agents such as cationic resins, lanolin derivatives, cholesterol, pantothenic acid and betaine. The said constituents are used in amounts commonly employed for such purposes, for example the wetting agents and emulsifiers at a concentration of about 0.5 to 30 wt. % (based on the dye carrier composition), the thickeners in an amount of about 0.1 to 30 wt. % (based on the dye carrier composition) and the hair-care agents at a concentration of about 0.1 to 5 wt. % (based on the dye carrier composition).

The pH of the ready-to-use colorant is usually about 3 to 11 and preferably about 1 to 6, a pH of 1 to 3 being particularly preferred. The pH of the ready-to-use colorant reached during the mixing of component (A1) containing the compounds of formula (I) and/or (Ia)/(Ib) with the electrophile-containing component (A2) depends on the pH of components (A1) and (A2) and on the mixing ratio of these two components. If necessary, after components (A1) and (A2) are mixed, the pH of the ready-to-use colorant can be adjusted to the desired level by use of an alkalinizing agent or an acid.

To adjust the-pH of the ready-to-use colorant and of components (A1) and (A2), an alkalinizing agent, for example an alkanolamine, alkylamine, alkali metal hydroxide, ammonium hydroxide, alkali metal carbonate or ammonium carbonate, or an acid, for example, lactic, acetic, tartaric, phosphoric, hydrochloric, citric, ascorbic or boric acid can be used.

The ready-to-use colorant is prepared just before use by mixing components (A1) containing the compounds of formulas (I) and/or (Ia)/(Ib) with component (A2) containing the electrophilic compound (optionally with addition of an alkalinizing agent or an acid) and is then applied to the fibers. Depending on the depth of shade desired, this mixture is allowed to act on the fibers for 5 to 60 minutes, preferably 15 to 30 minutes, at a temperature of 20° to 50° C. and particularly at 30° to 40° C. The fibers are then rinsed with water and optionally washed with a shampoo.

The colorant of the invention gives rise to a wide range of color shades from yellow-red to blue and brown-black, with outstanding fastness properties, with a gentle, uniform and lasting coloring of the fibers, particularly keratin fibers, for example human hair, being achieved even without the addition of oxidants.

Surprisingly, the colorations thus obtained can at any time be removed completely, rapidly and in gentle fashion by use of a reducing agent.

Another object of the present invention therefore is a method of coloring and later decolorizing keratin fibers, for example wool, silk or hair and particularly human hair, whereby the fibers are colored with a colorant (A) of the invention and then, at any desired time, again decolorized by use of a decolorizing agent (B), component (B) containing as the decolorizing substance at least one sulfite, for example ammonium sulfite, alkali metal sulfite or alkaline earth metal sulfite and particularly sodium sulfite or ammonium sulfite.

The total amount of sulfites in component (B) is about 0.1 to 10 wt. % and preferably about 2 to 5 wt. %.

The agent for decolorizing the fibers colored with colorant (A) (in the following referred to as “decolorizing agent”) can be in the form of an aqueous or aqueous-alcoholic solution, or a gel, cream, emulsion or foam, the decolorizing agent being packaged as a one-component or multicomponent preparation. The decolorizing agent can be packaged as a powder to protect against dusting or also in the form of tablets—including effervescent tablets—or as a granulate. From such forms, the decolorizing agent is then prepared before use by adding cold or warm water, optionally with addition of one or more of the following additives or auxiliary agents. It is, however, also possible for these additives and auxiliary agents (provided they are used as solids) to be already present in the decolorizing powder or decolorizing granulate or effervescent tablet. If necessary, dusting can be prevented by wetting the powder with an oil or wax.

The decolorizing agent can contain other additives and auxiliary agents, for example solvents such as water, lower aliphatic alcohols, for example ethanol, n-propanol and isopropanol, glycol ethers or glycols such as glycerol and particularly 1,2-propanediol; moreover wetting agents or emulsifiers from the classes of anionic, cationic, amphoteric or nonionic surface-active substances such as the fatty alcohol sulfates, ethoxylated fatty alcohol sulfates, alkyl sulfonates, alkylbenzenesulfonates, alkyltrimethylammonium salts, alkylbetaines, ethoxylated fatty alcohols, ethoxylated nonylphenols, fatty alkanolamides, ethoxylated fatty alcohols, ethoxylated nonylphenols, fatty alkanolamides [sic—Translator], ethoxylated fatty esters, furthermore thickeners such as the higher fatty alcohols, starch or cellulose derivatives, perfumes, hair pretreatment agents, conditioners, hair-swelling agents, preservatives, vaseline, paraffin oil and fatty acids as well as hair care agents such as cationic resins, lanolin derivatives, cholesterol, pantothenic acid and betaine.

The pH of the decolorizing agent is between about 3 and 8, and particularly between about 4 and 7. If necessary, the desired pH can be achieved by addition of a suitable acid, for example an α-hydroxycarboxylic acid such as lactic acid, tartaric acid, citric acid or malic acid, moreover phosphoric acid, acetic acid, glycolic acid, salicylic acid, glutathione or gluconolactone, or of an alkalinizing agent such as an alkanolamine, alkylamine, alkali metal hydroxide, ammonium hydroxide, alkali metal carbonate, ammonium carbonate or alkali metal phosphate.

The duration of exposure to the decolorizing agent is from 5 to 60 minutes and particularly from 15 to 30 minutes, depending on the color to be decolorized and on the temperature (about 20° to 50° C.), the decolorizing process being accelerated by supplying heat. At the end of the period of exposure to the decolorizing agent, the hair is rinsed with water and optionally washed with a shampoo.

Although component (B) is particularly well suited for decolorizing keratin fibers, particularly human hair, colored with colorant (A), component (B) can in principle also be used for decolorizing other natural or synthetic fibers colored with colorant (A), for example cotton, viscose, nylon or cellulose acetate.

The following examples are intended to explain the subject matter more closely without limiting its scope.

### EXAMPLES

### Examples 1.1 to 7.2

### Hair Colorants

Component A1

Compound of formula (1)/(Ia)/(Ib); amount as per Tables 1-7

| | | |
|---|---|---|
| Cetylstearyl alcohol | 12.0 | g |
| Polyethylene glycol octadecyl ether (Brij ® 78P, by Fluka) | 2.8 | g |
| Ethanol | 24.8 | g |
| Water, demineralized | to 100.0 | g |

Cetylstearyl alcohol was melted at 80° C. The compound of formula (I)/(Ia)/(Ib) was added to a hot (70° C.) mixture of polyethylene glycol octadecyl ether, water and ethanol and dissolved by stirring. This hot mixture was then added to the melted cetylstearyl alcohol and the resulting mixture was stirred until a cream formed. When necessary, the pH of the cream was adjusted to pH 2-3 with 90% lactic acid.

Component A2

Carbonyl compound/imine compound or
compound of formula (lla)/(llb); amount as per Tables 1-7

| | | |
|---|---|---|
| Cetylstearyl alcohol | 12.0 | g |
| Polyethylene glycol octadecyl ether (Brij ® 78P, by Fluka) | 2.8 | g |
| Ethanol | 24.8 | g |
| Water, demineralized | to 100.0 | g |

Variant 1: Carbonyl/Imine Compounds

Cetylstearyl alcohol was melted at 80° C. The polyethylene glycol octadecyl ether, water and the carbonyl compound dissolved in ethanol were heated to 80° C. This mixture was added to the melted cetylstearyl alcohol and stirred until a cream formed. When necessary, the pH of the cream was adjusted to 9-10 at room temperature by adding 20% aqueous monoethanolamine (in situ preparation of the imine compound).

Variant 2: Compounds of Formula (IIa)/(IIb)

Cetylstearyl alcohol was melted at 80° C. The polyethylene glycol octadecyl ether and 95% of the water were heated to 80° C. and added to the melted cetylstearyl alcohol and stirred until a cream formed. The compound of formula (IIa)/(IIb) together with the ethanol and the remainder of the water were then added at room temperature.

Component A1 and component A2 were mixed with one another in a 1:1 ratio. When necessary, the pH of the resulting mixture was adjusted to the pHₘ indicated in the examples by addition of a 90% lactic acid solution or a 20% monoethanolamine solution. The ready-to-use hair colorant thus obtained was applied to the hair and uniformly distributed with a brush. After an exposure time of 30 min at 40° C., the hair was washed with a shampoo, rinsed with luke-warm water and then dried.

The hair could at any time (for example after several days or weeks) be completely decolorized within 20 min at 40° C. by use of an acidic (pH 5) 5% sodium sulfite solution (component B).

The coloring and decolorizing results are summarized in the following Tables 1-7.

The L*a*b* color values given in the examples were determined with a Chromameter 11 color meter supplied by Minolta. The L value indicates the brightness (i.e., the lower the L value, the higher is the color intensity), the a value being a measure of the red content (i.e., the higher the a value, the higher is the red content). The b value is a measure of the blue content of the color, i.e., the more negative the b value the higher is the blue content.

Unless otherwise indicated, all percentages in the present patent application are by weight.

TABLE 1
Coloring Results

| | | Color Shade | | | | |
|---|---|---|---|---|---|---|
| | | After | | | | |
| | Component A1 - pH 2-3 | Dyeing/ | | Color Values | | |
| No. | Component A2 - pH 4-5 | Decoloration | | L | a | b |
| 1.1 | A1) 7-Hydroxy-4-methyl-2- | brown-red | untreated hair: | 63.30 | −0.48 | +10.40 |
| | phenyl-1-benzopyrylium | | after dyeing: | 26.64 | +21.25 | +13.13 |
| | chloride: 3.14 g | | | | | |
| | A2) 4-Hydroxy-3-methoxy- | after deco-: | slightly orange | | | |
| | benzaldehyde: 1.75 g | loration | | | | |
| | pHₘ 2.3 | | | | | |
| 1.2 | A1) 7-Hydroxy-4-methyl-2- | brown | untreated hair: | 63.30 | −0.48 | +10.40 |
| | phenyl-1-benzopyrylium | | after dyeing | 24.06 | +9.79 | +7.89 |
| | chloride: 3.14 g | | | | | |
| | A2) 3,5-dimethoxy-4-hy- | after deco- | slightly orange | | | |
| | droxybenzaldehyde: 2.10 g | loration | | | | |
| | pHₘ 2.8 | | | | | |
| 1.3 | A1) 7-Hydroxy-4-methyl-2- | green | untreated hair: | 63.30 | −0.48 | +10.40 |
| | phenyl-l-benzopyrylium | | after dyeing: | 26.54 | +4.58 | +13.39 |
| | chloride: 3.14 g | | | | | |
| | A2) 4-(dimethylamino)- | after deco- | slightly yellow | | | |
| | benzaldehyde: 1.72 g | loration | | | | |
| | pHₘ 2.6 | | | | | |
| 1.4 | A1) 7-Hydroxy-4-methyl-2- | dark-red | untreated hair: | 63.30 | −0.48 | +10.40 |
| | phenyl-1-benzopyrylium | | after dyeing: | 22.55 | +33.09 | +2.02 |
| | chloride: 3.14 g | | | | | |
| | A2) 2-Chloro-1-ethyl- | | | | | |
| | quinolinium tetrafluoro- | | | | | |
| | borate: 3.21 g | | | | | |
| | pHₘ 7.9 | | | | | |
| 1.5 | A1) 7-Hydroxy-4-methyl-2- | brown | untreated hair: | 63.30 | −0.48 | +10.40 |
| | phenyl-l-benzopyrylium | | after dyeing: | 33.88 | +12.12 | +22.80 |
| | chloride: 3.14 g | | | | | |
| | A2) 4-Dimethylamino-1- | | | | | |
| | naphthaldehyde: 2.29 g | | | | | |
| | pHₘ 2.4 | | | | | |

TABLE 2
Coloring Results

| | Component A1 - pH 2-3 | Color Shade | | Color Values | | |
|---|---|---|---|---|---|---|
| No. | Component A2 - pH 9-10 | After Dyeing | | L | a | b |
| 2.1 | A1) 7-Methoxy-4-methyl-2- | brown | untreated hair: | 63.30 | −0.48 | +10.40 |
| | (3,4,5-trimethoxyphenyl)-1- | | after dyeing: | 39.76 | +8.20 | +10.20 |
| | benzopyrylium chloride: | | | | | |
| | 4.33 g | | | | | |
| | A2) 4-Hydroxy-3-methoxy- | | | | | |
| | benzaldehyde: 1.75 g | | | | | |
| | pHₘ 2.9 | | | | | |
| 2.2 | A1) 7-Methoxy-4-methyl-2- | gray | untreated hair: | 63.30 | −0.48 | +10.40 |
| | (3,4,5-trimethoxyphenyl)-1- | | after dyeing | 37.77 | +1.52 | +4.28 |
| | benzopyrylium chloride: | | | | | |
| | 4.33 g; | | | | | |
| | A2) 3,5-Dimethoxy-4-hy- | | | | | |
| | droxybenzaldehyde: 2.10 g; | | | | | |
| | pHₘ 2.9 | | | | | |
| 2.3 | A1) 7-Methoxy-4-methyl-2- | green | untreated hair: | 63.30 | −0.48 | +10.40 |
| | (3,4,5-trimethoxyphenyl)-1- | | after dyeing: | 44.24 | −26.15 | +13.38 |
| | benzopyrylium chloride: | | | | | |
| | 4.33 g; | | | | | |
| | A2) 4-(Dimethylamino)- | | | | | |
| | benzaldehyde: 1.72 g | | | | | |
| | pHₘ 2.6 | | | | | |

TABLE 3
Coloring Results

| | Component A1 - pH 2-3 | Color Shade | | Color Values | | |
|---|---|---|---|---|---|---|
| No. | Component A2 - pH 4-5 | After Dyeing | | L | a | b |
| 3.1 | A1) 7-Methoxy-4-methyl-2- | red | untreated hair: | 63.30 | −0.48 | +10.40 |
| | (3,4,5-trimethoxyphenyl)-l- | | after dyeing: | 41.87 | +35.12 | +1.15 |
| | benzopyrylium chloride: | | | | | |
| | 4.33 g | | | | | |
| | A2) 4-Chloro-1-ethylquino- | | | | | |
| | linium tetrafluoroborate: | | | | | |
| | 3.21 g | | | | | |
| | pHₘ 2.5 | | | | | |
| 3.2 | A1) 7-Methoxy-4-methyl-2- | red | after dyeing | 38.49 | +29.18 | +5.46 |
| | (3,4,5-trimethoxyphenyl)-1- | red | | | | |
| | benzopyrylium chloride: | | | | | |
| | 4.33 g | | | | | |
| | A2) 4-Chloro-1-ethylquino- | | | | | |
| | linium tetrafluoroborate: | | | | | |
| | 3.21 g | | | | | |
| | pHₘ 9.7 | | | | | |

TABLE 4
Coloring Results

| | Component A1 - pH 2-3 | Color Shade | | Color Values | | |
|---|---|---|---|---|---|---|
| No. | Component A2 - pH 4-5 | After Dyeing | | L | a | b |
| 4.1 | A1) 5,7-Dihydroxy-4-methyl- | brown | untreated hair: | 63.30 | −0.48 | +10.40 |
| | 2-phenyl-1-benzopyrylium | | after dyeing: | 32.59 | +29.39 | +20.37 |
| | chloride: 3.32 g | | | | | |
| | A2) 4-Hydroxy-3-methoxy- | | | | | |
| | benzaldehyde: 1.75 g | | | | | |
| | pHₘ 2.5 | | | | | |
| 4.2 | A1) 5,7-Dihydroxy-4-methyl- | brown | untreated hair: | 63.30 | −0.48 | +10.40 |
| | 2-phenyl-1-benzopyrylium | | after dyeing: | 26.28 | +11.68 | +9.74 |
| | chloride: 3.32 g | | | | | |
| | A2) 4-(Dimethylamino)- | | | | | |
| | benzaldehyde: 1.72 g | | | | | |
| | pHₘ 2.45 | | | | | |

TABLE 5
Coloring Results

| | Component A1 - pH 2-3 | Color Shade | | Color Values | | |
|---|---|---|---|---|---|---|
| No. | Component A2 - pH 4-5 | After Dyeing | | L | a | b |
| 5.1 | A1) 5,7-Dihydroxy-4-methyl- | dark-red | untreated hair: | 63.30 | −0.48 | +10.40 |
| | 2-phenyl-1-benzopyrylium | | after dyeing: | 30.91 | +13.65 | +13.92 |
| | chloride: 3.32 g | | | | | |
| | A2) 4-Chloro-1-ethylquino- | | | | | |
| | linium tetrafluoroborate: | | | | | |
| | 3.21 g | | | | | |
| | pHₘ 2.35 | | | | | |
| 5.2 | A1) 5,7-Dihydroxy-4-methyl- | dark-red | untreated hair: | 63.30 | −0.48 | +10.40 |
| | 2-phenyl-1-benzopyrylium | | after dyeing: | 26.07 | +33.70 | +4.33 |
| | chloride: 3.32 g | | | | | |
| | A2) 4-Chloro-1-ethylquino- | | | | | |
| | linium tetrafluoroborate: | | | | | |
| | 3.21 g | | | | | |
| | pHₘ 7.1 | | | | | |

TABLE 6
Coloring Results

| | Component A1 - pH 2-3 | Color Shade | | Color Values | | |
|---|---|---|---|---|---|---|
| No. | Component A2 - pH 4-5 | After Dyeing | | L | a | b |
| 6.1 | A1) 7-Hydroxy-2,4-dimethyl- | green | untreated hair: | 63.30 | −0.48 | +10.40 |
| | 1-benzopyrylium chloride: | | after dyeing: | 47.81 | −19.37 | +30.10 |
| | 2.42 g | | | | | |
| | A2) 4-(Dimethylamino)- | | | | | |
| | benzaldehyde: 3.43 g | | | | | |
| | pHₘ 2.6 | | | | | |
| 6.2 | A1) 7-Hydroxy-2,4-dimethyl- | green | untreated hair: | 63.30 | −0.48 | +10.40 |
| | 1-benzopyrylium chloride: | | after dyeing: | 52.63 | −13.70 | +37.00 |
| | 2.42 g | | | | | |
| | A2) 4-(Dimethylamino)- | | | | | |
| | benzaldehyde: 3.43 g | | | | | |
| | pHₘ 7.5 | | | | | |
| 6.3 | A1) 7-Hydroxy-2,4-dimethyl- | brown | untreated hair: | 63.30 | −0.48 | +10.40 |
| | 1-benzopyrylium chloride: | | after dyeing: | 49.80 | +13.28 | +40.32 |
| | 2.42 g | | | | | |
| | A2) 4-Hydroxy-3-methoxy- | | | | | |
| | benzaldehyde: 3.50 g; | | | | | |
| | pHₘ 7.5 | | | | | |
| 6.4 | A1) 7-Hydroxy-2,4-dimethyl- | brown | untreated hair | 63.30 | −0.48 | +10.40 |
| | 1-benzopyrylium chloride: | | after dyeing | 46.81 | +3.22 | +38.83 |
| | 2.42 g | | | | | |
| | A2) 3,5-Dimethoxy-4-hy- | | | | | |
| | droxy benzaldehyde: 4.19 g | | | | | |
| | pHₘ 7.0 | | | | | |
| 6.5 | A1) 7-Hydroxy-2,4-dimethyl- | dark-red | untreated hair | 63.30 | −0.48 | +10.40 |
| | 1-benzopyrylium chloride: | | after dyeing | 30.24 | +54.22 | +15.23 |
| | 2.42 g | | | | | |
| | A2) 4-Chloro-1-ethylquino- | | | | | |
| | linium tetrafluoroborate: | | | | | |
| | 6.43 g | | | | | |
| | pHₘ 7.9 | | | | | |

TABLE 7
Coloring Results

| | Component A1 - pH 2-3 | Color Shade | | Color Values | | |
|---|---|---|---|---|---|---|
| No. | Component A2 - pH 4-5 | After Dyeing | | L | a | b |
| 7.1 | A1) 5,7-Dihydroxy-2,4-di- | green | untreated hair: | 63.30 | −0.48 | +10.40 |
| | methyl-1-benzopyrylium | | after dyeing: | 57.52 | −10.31 | +9.96 |
| | chloride: 2.61 g | | | | | |
| | A2) 4-(Dimethylamino)- | | | | | |
| | benzaldehyde: 3.43 g | | | | | |
| | pHₘ 2.6 | | | | | |
| 7.2 | A1) 5,7-Dihydroxy-2,4-di- | dark-red | untreated hair: | 63.30 | −0.48 | +10.40 |
| | methyl-1-benzopyrylium | | after dyeing: | 40.79 | +61.98 | +7.49 |
| | chloride: 2.61 g | | | | | |
| | A2) 4-Chloro-1-ethyl- | | | | | |
| | quinolinium tetrafluoro- | | | | | |
| | borate: 6.43 g | | | | | |
| | pHₘ 2.4 | | | | | |

1. Agent for coloring fibers that is obtained by mixing two components—if necessary with addition of an alkalinizing agent or an acid—characterized in that one component (component A2) contains at least one electrophilic compound and the other component (component A1) contains at least one 1-benzopyran of formula (Ia)/(Ib) or a 1-benzopyrylium derivative of formula (I), 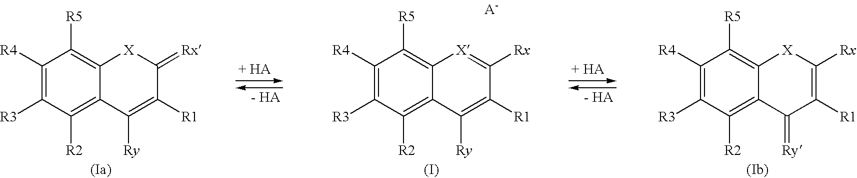
wherein:
X denotes an oxygen atom, sulfur atom or selenium atom;
Rx and Ry independently of each other denote a hydrogen atom, an aryl group, a C₁-C₈-alkyl group, a C₁-C₈-monohydroxyalkyl group, a C₁-C₈-polyhydroxyalkyl group, a C₁-C₈-alkoxy-(C₁-C₈)-alkyl group, a halogen atom, an —O—COR^{a} group, an —S—COR^{a} group, an —SR^{a} group, a heteroaryl group, a phenyl group, a benzyl group, an —OCH₂-aryl group or a —CH₂-Rz group;
Rx′ and Ry′ independently of each other denote a —CH-Rz group;
Rz denotes a hydrogen atom, an optionally substituted phenyl group, an optionally substituted naphthyl group, an optionally substituted aromatic carbon ring or heterocyclic ring, a C₁-C₈-alkyl group, a C₁-C₈-monohydroxyalkyl group, a C₁-C₈-polyhydroxyalkyl group, a C₁-C₈-alkoxy-(C₁-C₈)-alkyl group, a halogen atom, an —OCOR^{a} group, a nitro group, a cyano group, a —CO—R^{a} group, a —CO—OR^{a} group, a —CO—OCF₃ group, a —CONHR^{a} group, a —CO—N(R^{a})₂ group, an —SO₂—NH₂ group, an —SO₂—NHR^{a} group, an —SO₂—N(R^{a})₂ group or an —SO₂—OR^{a} group, wherein R^{a} stands for a hydrogen atom, an optionally substituted aromatic carbon ring or heterocyclic ring or a (C₁-C₆)-alkyl group; provided that the tautomeric formula (Ia) is valid only when Rx is a —CH₂-Rz group, and the tautomeric formula (Ib) is valid only when Ry is a —CH₂-Rz group, and Rz in Rx and Rx′ or Ry and Ry′ always has the same meaning;
R1, R2, R3, R4 and R5 independently of each other denote a hydrogen atom, a straight-chain or branched (C₁-C₄)-alkyl group, a (C₁-C₄)-hydroxyalkyl group, a hydroxyl group, a methoxy group, a benzyl group, a halogen atom, a nitro group, a nitroso group, a cyano group, a trifluoromethyl group, a —CHO group, a —COR^{b} group, a —COOH group, a —CO₂R^{b} group, an —OCOR^{b} group, an —OCH₂-aryl group, an —SO₂NH₂ group, an —SO₂CHF₂ group, an —SO₂CF₃ group, an —SO₂NH₂ group [sic—Translator], an —SO₂NHR^{b} group, an —SO₂N(R^{b})₂ group, an —SO₂R^{b} group, an —NH₂ group, an —NH₃⁺ group, an —NHR^{b} group, an —NH₂R^{b+} group, an —N(R^{b})₂ group, an —N(R^{b})₃⁺ group, an —NHCOR^{b} group, an —NHCOOR^{b} group, a —CH₂NH₂ group, a —CH₂NHR^{b} group, a —CH₂N(R^{b})₂ group, a —CO₂CF₃ group or a —PO(OR^{b})₂ group, wherein R^{b} stands for a hydrogen atom, an optionally substituted aromatic carbon ring or heterocyclic ring or a (C₁-C₆)-alkyl group); and
A⁻ denotes an anion of an organic or inorganic acid.

2. Agent according to claim 1, characterized in that the compound of formula (I), (Ia) and (Ib) is selected from among 7-hydroxy-4-methylene-2-phenyl-4H-1-benzopyran; 7-methoxy-4-methylene-2-(3,4,5-trimethoxyphenyl)-4H-1-benzopyran; 5,7-dihydroxy-4-methylene-2-phenyl-4H-1-benzopyran; 5,7-dimethoxy-4-methylene-2-phenyl-4H-1-benzopyran; 4-methylene-4H-1-benzopyran; 4-methylene-2-phenyl-4H-1-benzopyran; 7-amino-4-methylene-2-phenyl-4H-1-benzopyran; 7-dimethylamino-4-methylene-2-phenyl-4H-1-benzopyran; 4-methylene-7-nitro-2-phenyl-4H-1-benzopyran; 2-methylene-2H-1-benzopyran; 2-methylene-4-phenyl-2H-1-benzopyran; 2-methylene-4-methyl-2H-1-benzopyran and 4-methylene-2-methyl-4H-1-benzopyran and the salts thereof.

3. Agent according to claim 1, characterized in that the compound of formula (I), (Ia) and (Ib) is selected from among 7-hydroxy-4-methyl-2-phenyl-1-benzopyrylium chloride; 7-methoxy-4-methyl-2-(3,4,5-trimethoxyphenyl)-1-benzopyrylium chloride; 5,7-dihydroxy-4-methyl-2-phenyl-1-benzopyrylium chloride; 5,7-dimethoxy-4-methyl-2-phenyl-1-benzopyrylium chloride; 4-methyl-1-benzopyrylium chloride; 4-methyl-2-phenyl-1-benzopyrylium chloride; 7-amino-4-methyl-2-phenyl-1-benzopyrylium chloride; 7-dimethylamino-4-methyl-2-phenyl-1-benzopyrylium chloride; 4-methyl-7-nitro-2-phenyl-1-benzopyrylium chloride; 2-methyl-1-benzopyrylium chloride; 2-methyl-4-phenyl-1-benzopyrylium chloride; 7-hydroxy-2,4-dimethyl-1-benzopyrylium chloride; 5,7-dihydroxy-2,4-dimethyl-1-benzopyrylium chloride; 7-dimethylamino-2,4-dimethyl-1-benzopyrylium [sic—Translator] and 2,4-dimethyl-1-benzopyrylium chloride.

4. Agent according to one of claims 1 to 3, characterized in that the electrophilic compound is selected from among carbonyl compounds, imine compounds and substituted 1-alkylquinolinium derivatives of formulas (IIa) and (IIb) 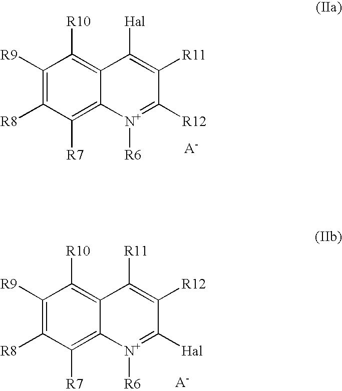
wherein
group R6 denotes a straight-chain or branched C₁-C₈-alkyl group, a C₁-C₈-monohydroxyalkyl group, a C₂-C₈-polyhydroxyalkyl group, a C₁-C₈-alkoxy-(C₁-C₈)-alkyl group or a thio-(C₁-C₈)-alkyl group;
R7, R8, R9, R10, R11 and R12 groups independently of each other denote a hydrogen atom, a straight-chain or branched C₁-C₄-alkyl group, a straight-chain or branched C₁-C₄ hydroxyalkyl group, a hydroxyl group, a methoxy group, an ethoxy group, a benzyl group, a halogen atom, a nitro group, a nitroso group, a cyano group, a trifluoromethyl group, a —CHO group, a —CORC group, a —COOH group, a —CO₂R^{c} group, an —OCOR^{c} group, an —O—SO₂CF₃ group, an —OCH₂-aryl group, an —SO₂NH₂ group, an —SO₂CHF₂ group, an —SO₂CF₃ group, an —SO₂NH₂ group, an —SO₂NHR^{c} group, an —SO₂N(R^{c})₂ group, an —SO₂R^{c} group, an —NH₂ group, an —(NH₃)⁺ group, an —NHR^{c} group, an —(NH₂R^{c})⁺ group, an —N(R^{c})₂ group, an —[N(R^{c})₃]⁺ group, an —NHCOR^{c} group, an —NHCOOR^{c} group, a —CH₂NH₂ group, a —CH₂NHR^{c} group, a —CH₂N(R^{c})₂ group, a —CO₂CF₃ group or a —PO(OR^{c})₂ group, wherein R^{c} stands for a hydrogen atom, an optionally substituted aromatic carbon ring or heterocyclic ring or a (C₁-C₆)-alkyl group;
Hal denotes an iodine atom, bromine atom, chlorine atom or a methoxy group, ethoxy group, CF₃—SO₂—O— group, aryl-SO₂—O— group or a (SO₃)⁻ group, with a chlorine atom and ethoxy group being preferred, and
A⁻ denotes the anion of an organic or inorganic acid.

5. Agent according to claim 4, characterized in that the carbonyl compound or imine compound is selected from among 4-hydroxy-3-methoxybenzaldehyde, 3-hydroxy-4-methoxybenzaldehyde, 3,4-dihydroxybenzaldehyde, 4-hydroxybenzaldehyde, 3,5-dimethoxy-4-hydroxybenzaldehyde, 4-dimethylaminobenzaldehyde, 4-methyl-5-imidazolcarboxaldehyde, 4-dimethylaminocinnamaldehyde, 4-hydroxy-2-methoxybenzaldehyde, 3,5-dimethyl-4-hydroxybenzaldehyde, 4-dimethylamino-2-methoxybenzaldehyde, 2-hydroxybenzaldehyde, 4-hydroxy-1-naphthaldehyde, 4-methoxy-1-naphthaldehyde, 4-dimethylamino-1-naphthaldehyde, 4′-hydroxybiphenyl-1-carbaldehyde, 2-hydroxy-3-methoxybenzaldehyde, 2,4-dihydroxybenzaldehyde, 3,4-dihydroxybenzaldehyde, 2,5-dihydroxybenzaldehyde, 2,3,4-trihydroxybenzaldehyde, 3,4,5-trihydroxybenzaldehyde, 2,4,6-trihydroxybenzaldehyde, 2,4-dimethoxybenzaldehyde, 2,3-dimethoxybenzaldehyde, 2,5-dimethoxybenzaldehyde, 3,5-dimethoxybenzaldehyde, 3,4-dimethoxybenzaldehyde, indole-3-carbaldehyde, benzene-1,4-dicarbaldehyde, 4-ethoxybenzaldehyde, 2-methyl-1,4-naphthoquinone, 4-carboxybenzaldehyde, 4-hydroxy-3-methoxycinnamaldehyde, 3, 5-dimethoxy-4-hydroxycinnamaldehyde, 3-methoxy-4-(1-pyrrolidinyl)benzaldehyde, 4-diethylamino-3-methoxybenzaldehyde, 1,2-phthalic dialdehyde, pyrrole-2-aldehyde, thiophene-2-aldehyde, thiophene-3-aldehyde, chromone-3-carboxaldehyde, 6-methyl-4-keto-1 (4H)-benzopyran-3-carbaldehyde, N-methylpyrrole-2-aldehyde, 5-methylfurfural, 6-hydroxychromene-3-carboxaldehyde, 6-methylindole-3-carboxaldehyde, 4-dibutylaminobenzaldehyde, N-ethylcarbazole-3-aldehyde, 4-diethylamino-2-hydroxybenzaldehyde, 3,4-dimethoxy-5-hydroxybenzaldehyde, 5-[4-(diethylamino)phenyl]-2,4-pentadienal, 2,3-thiophenedicarboxaldehyde, 2, 5-thiophenedicarboxaldehyde, 2-methoxy-1-naphthaldehyde, 3-ethoxy-4-hydroxybenzaldehyde, 2-nitrobenzaldehyde, 3-nitrobenzaldehyde and 4-nitrobenzaldehyde, 4-{[(2-hydroxyethyl)imino]methyl}-2-methoxyphenol, 5-{[(2-hydroxyethyl)imino]methyl}-2-methoxyphenol, 2,6-dimethoxy-4-{[(2-hydroxyethyl)imino]methyl}phenol, 4-{[(2-hydroxyethyl)imino]methyl}-phenol, 1,2-dihydroxy-4-{[(2-hydroxyethyl)imino]methyl}benzene, N,N-dimethyl-4-{[(2-hydroxyethyl)imino]methyl}aniline, 1,2-dihydroxy-3-{[(2-hydroxyethyl)imino]methyl}benzene, 4-{[(3-hydroxypropyl)imino]methyl}phenol, 2,6-dimethoxy-4-{[(3-hydroxypropyl)imino]methyl}phenol, 4-{[(2,3-dihydroxypropyl)imino]methyl}phenol, 2,6-dimethoxy-4-{[(2,3-dihydroxypropyl)imino]methyl}phenol, 2-[(4-hydroxybenzylidene)amino]propane-1,3-diol, 2-[(4-hydroxy-3,5-dimethoxybenzylidene)amino]propane-1,3-diol, 4-{[(2-hydroxy-2-phenylethyl)imino]methyl}phenol, 2,6-dimethoxy-4-{[(1-phenyl-2-hydroxyethyl)imino]methyl}phenol, 4-{[(2-hydroxyphenyl)imino]methyl}-phenol, 2,6-dimethoxy-4-{[(2-hydroxyphenyl)imino]methyl}phenol, 5-guanidino-2-[(4-hydroxybenzylidene)amino]pentanoic acid, 2-[(4-dimethylaminonaphthalen-1-ylmethylene)amino]ethanol, 5-guanidino-2-[(4-hydroxy-3,5-dimethoxybenzylidene)-amino]pentanoic acid, 2-[(4-hydroxy-3,5-dimethoxybenzylidene)amino]-3-(3H-imidazol-4-yl)propanoic acid, 2-[(4-hydroxybenzylidene)amino]-3-(3H-imidazol-4-yl)propanoic acid, 2-[(4-hydroxy-3,5-dimethoxybenzylidene)amino]-3-(1H-indol-3-yl)propanoic acid, 2-[(4-hydroxybenzylidene)amino]-3-(1H-indol-3-yl)propanoic acid, 2-{[(2-hydroxy-ethyl)imino]methyl}phenol, 1,2-dihydroxy-3-{[(2-hydroxyethyl)imino]-methyl}benzene, 1,2,3-trihydroxy-4-{[(2-hydroxyethyl)imino]methyl}benzene, 1,2,3,4-tetrahydroxy-5-{[(2-hydroxyethyl)imino]methyl}benzeneand 1,2,4-trihydroxy-4-{[(2-hydroxyethyl)imino]methyl}benzene.

6. Agent according to claim 4 or 5, characterized in that the carbonyl compound is prepared in situ by enzymatic oxidation of the corresponding aryl alcohols or benzyl alcohols with the aid of one or more suitable oxidation enzymes.

7. Agent according to claim 4, characterized in that the substituted 1-alkylquinolinium compound of formulas (IIa) and (IIb) is selected from among 4-chloro-1-ethylquinolinium salts, 4,7-dichloro-1-ethylquinolinium salts, 4-chloro-1-ethyl-7-trifluoromethylquinolinium salts, 4-chloro-1-ethyl-6-nitroquinolinium salts, 4-chloro-1-methylquinolinium salts, 4-methoxy-1-methylquinolinium salts, 4-ethoxy-1-methylquinolinium salts, 4-ethoxy-1-ethylquinolinium salts, 4-iodo-1-methylquinolinium salts, 4-chloro-1-methyl-2-phenylquinolinium salts, 4-chloro-1-methyl-3-[(methylphenylamino)sulfonyl]quinolinium salts, 4-chloro-2-{{[(4-dimethylamino)-phenyl]imino}methyl}-6-methoxy-1-methylquinolinium salts, 4-chloro-1-ethyl-3-[(phenylamino)sulfonyl]quinolinium salts, 4-chloro-6-dimethylcarbamoyl-1-methylquinolinium salts, 4-chloro-1-ethyl-6-sulfamoylquinolinium salts, 4-chloro-1-ethyl-6-formylquinolinium salts, 4-chloro-1-ethyl-7-nitroquinolinium salts, 4-chloro-1-ethyl-7-methoxyquinolinium salts, 2-chloro-1-methylquinolinium salts, 2,6-dichloro-1-methylquinolinium salts and 2-chloro-1-methyl-4-trifluoromethanesulfonyloxy-quinolinium salts.

8. Agent according to claim 7, characterized in that the substituted 1-alkylquinolinium compound of formulas (IIa) and (IIb) is selected from among 4-chloro-1-ethylquinolinium tetrafluoroborate, 4,7-dichloro-1-ethylquinolinium tetrafluoroborate, 4-chloro-1-ethyl-7-trifluoromethylquinolinium [sic—Translator], 4-chloro-1-ethyl-6-nitroquinolinium tetrafluoroborate, 4-chloro-1-methylquinolinium chloride, 4-methoxy-1-methylquinoliniumtetrafluoroborate, 4-iodo-1-methylquinolinium iodide, 4-chloro-1-methyl-2-phenylquinoliniumtetrafluoroborate,4-chloro-1-methyl-3-[(methylphenylamino)sulfonyl]quinolinium methylsulfate, 4-chloro-2-{{[4-(dimethylamino)-phenyl]imino}methyl}-6-methoxy-1-methylquinolinium chloride, 4-chloro-1-ethyl-3-[(phenylamino)sulfonyl]quinolinium tetrafluoroborate, 4-chloro-6-dimethylcarbamoyl-1-methylquinolinium tetrafluoroborate, 4-chloro-1-ethyl-6-sulfamoylquinolinium tetrafluoroborate, 4-chloro-1-ethyl-6-formylquinolinium tetrafluoroborate, 4-chloro-1-ethyl-7-nitroquinoliniumtetrafluoroborate,4-chloro-1-ethyl-7-methoxyquinoliniumtetrafluoroborate, 2-chloro-1-methylquinolinium tetrafluoroborate, 2,6-dichloro-1-methylquinolinium tetrafluoroborate and 2-chloro-1-methyl-4-trifluoromethanesulfonyloxyquinolinium tetrafluoroborate.

9. Agent according to one of claims 1 to 8, characterized in that the ready-to-use preparation (A) has a pH of 3 to 11.

10. Agent according to one of claims 1 to 9, characterized in that the compound of formula (I)/(Ia)/(Ib) is contained in component (A1) in a total amount of 0.02 to 20 wt. % and the electrophilic compound is contained in component A2 in a total amount of 0.02 to 20 wt. %.

11. Agent according to one of claims 1 to 1 0, characterized in that it contains the compound of formula (I)/(Ia)/(Ib) and the electrophilic compound in a total amount of 0.01 to 10 wt. % each, based on the ready-to-use preparation.

12. Agent according to one of claims 1 to 11, characterized in that the ready-to-use agent (A) as well as components (A1) and (A2) are in the form of a solution, emulsion, foam, cream or gel.

13. Agent according to one of claims 1 to 12, characterized in that component (A1) and/or (A2) contain additionally at least one direct dye from the group of acidic or basic dyes, nitro dyes, azo dyes, quinone dyes and triphenylmethane dyes.

14. Agent according to one of claims 1 to 13, characterized in that component (A1) and/or (A2) consist of several individual components.

15. Agent according to one of claims 1 to 14, characterized in that it is a hair colorant.

16. Method of temporarily coloring keratin fibers whereby the keratin fibers are colored with an agent according to one of claims 1 to 15 and then, at any future time, the resulting coloration is again removed by means of a sulfite-containing preparation (decolorizing agent).
